(19) United States
(12) Patent Application Publication
(10) Pub. No.: US 2025/0332428 A1
(43) Pub. Date: Oct. 30, 2025

(54) RECHARGE ALIGNMENT DETECTION FOR IMPLANTABLE DEVICE

(71) Applicant: Medtronic, Inc., Minneapolis, MN (US)

(72) Inventors: Andrew T. Fried, St. Paul, MN (US); Jacob P. Komarek, Fridley, MN (US); Charles M. Nowell, JR., Longwood, FL (US)

(21) Appl. No.: 19/188,419

(22) Filed: Apr. 24, 2025

Related U.S. Application Data
(60) Provisional application No. 63/639,503, filed on Apr. 26, 2024.

(51) Int. Cl. A61N 1/378; A61N 1/36; A61N 1/372; H02J 50/10; H02J 50/80; H02J 50/90

(52) U.S. Cl. CPC A61N 1/3787; A61N 1/36007; A61N 1/3605; A61N 1/37217; H02J 50/10; H02J 50/80; H02J 50/90

(57) ABSTRACT

Devices, systems, and techniques provide feedback to a patient to help the patient align a charging device to a medical device. In some examples, system may include a recharge coil configured to transfer energy to an implantable medical device (IMD) and detect metal loading and charging circuitry coupled to the recharge coil and configured to determine one or more electrical properties of the recharge coil during the transfer of energy. The system may also include processing circuitry configured to determine, based on the one or more electrical properties, a load on the recharge coil, compare the load on the recharge coil to one or more thresholds, and responsive to the load satisfying the threshold, perform an action associated with the transfer of energy to the IMD. An action may include displaying feedback regarding positioning of the charging device.

This application claims the benefit of and priority to U.S. Provisional Patent Application No. 63/639,503, filed Apr. 26, 2024, the entire contents of which is incorporated herein by reference.

### TECHNICAL FIELD

The disclosure relates to implantable medical devices, and more particularly to recharging of medical devices.

### BACKGROUND

Medical devices may be external or implanted and may be used to monitor patient signals such as cardiac activity, biological impedance and to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis and other conditions. In some examples, medical devices may include a rechargeable electrical power source, or may be powered directly by transmitting energy through tissue.

### SUMMARY

In general, the disclosure describes devices, systems, and techniques to provide feedback to a user to help improve alignment between a recharging device and a medical device. In some examples, a patient may hold or position a recharging device with respect to a rechargeable medical device that is implanted at a location in the patient. The energy transfer efficiency may depend on the alignment of a primary coil of the recharging device with a secondary coil of the implanted device. Therefore, feedback from the system to indicate to the user when and/or how to move the recharging device to improve energy transfer may reduce recharge time and/or reduce heating of the medical device (e.g., improved energy transfer efficiency).

A medical device, such as an implantable medical device (IMD) may deliver electrical stimulation therapy to the patient, and periodically need to receive recharge energy to replenish the battery, or a similar energy storage unit. Since the medical device, particularly when the medical device is an IMD, may be difficult to align with the recharge device, the recharge device or other device may provide feedback to the user to indicate that the recharge device needs to move. In some examples, the IMD may communicate energy transfer information back to the recharge device in order for the recharge device to determine how to provide feedback. However, this communication may occur infrequent during charging. Therefore, the recharge device may directly detect changes to loading on the recharge coil and use the loading to provide feedback to the user regarding when, or how, to move the recharge device with respect to the IMD.

In one example, this disclosure describes an external charging device, the device including a recharge coil configured to transfer energy to an implantable medical device (IMD) and detect metal loading, charging circuitry coupled to the recharge coil and configured to determine one or more electrical properties of the recharge coil during the transfer of energy, and processing circuitry configured to determine, based on the one or more electrical properties, a load on the recharge coil, compare the load on the recharge coil to one or more thresholds, and responsive to the load satisfying the threshold, perform an action associated with the transfer of energy to the IMD.

In another example, this disclosure describes a method including transferring, by a recharge coil, energy to an implantable medical device (IMD), wherein the recharge coil is configured to detect metal loading, determining, by charging circuitry coupled to the recharge coil, one or more electrical properties of the recharge coil during the transfer of energy, determining, by processing circuitry and based on the one or more electrical properties, a load on the recharge coil, comparing, by the processing circuitry, the load on the recharge coil to one or more thresholds, and responsive to the load satisfying the threshold, performing, by the processing circuitry, an action associated with the transfer of energy to the IMD.

In another example, this disclosure describes a non-transitory computer-readable medium including instructions that, when executed, controls the processing circuitry to control a recharge coil to transfer energy to an implantable medical device (IMD), wherein the recharge coil is configured to detect metal loading, wherein charging circuitry coupled to the recharge coil determines one or more electrical properties of the recharge coil during the transfer of energy, determine, based on the one or more electrical properties, a load on the recharge coil, compare the load on the recharge coil to one or more thresholds, and responsive to the load satisfying the threshold, perform an action associated with the transfer of energy to the IMD.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1A is a conceptual diagram illustrating a medical system of this disclosure that includes an implantable medical device located near a tibial nerve of a patient.

FIG. 1B is a conceptual diagram illustrating a medical system of this disclosure that includes an implantable medical device configured to deliver spinal cord stimulation to a patient.

FIG. 2 is a block diagram illustrating example components of the implantable medical device of FIGS. 1A and 1B.

FIG. 3 is a block diagram of an example external charging device of FIGS. 1A and 1B.

FIG. 4 is a flowchart illustrating an example operation of a recharging system for detecting metal load during energy transfer.

FIG. 5 is a flowchart illustrating an example operation of a recharging system for presenting an indication to a user based on metal load changes.

### DETAILED DESCRIPTION

Devices, system, and techniques configured to provide feedback to a patient to help the patient align a medical device to a location on their anatomy are described herein. In some examples, the patient may align a recharging device to a medical device that is implanted at the location on their anatomy. Proper alignment of the recharging device with the implanted device may improve energy transfer, which may, for example, recharge the implanted device more efficiently, generate less heat, and take less time. Less recharging time may be more convenient for the patient and improve user compliance with recharging requirements and therapy.

In the example of a rechargeable device, recharger coil alignment with an implantable device is desirable for improved recharging performance. In some examples, energy transfer efficiency is improved when the recharge coil, which may be included within the housing of the recharge device or within a wand coupled to the recharge device housing, is aligned with the secondary coil of the IMD. In some examples, a medical system may include a user interface to provide users feedback on recharge coupling to help the patient better align the recharger. The user interface may include a display screen that may provide a graphical indication of alignment. In some examples, the implant location may be near the posterior side of the patient, such as near the hip or spinal cord, which may make it awkward for some patients to align their recharger head with their implantable device. In some examples, a patient may establish good recharger alignment, but then during the course of recharging (which may take an hour or more), the recharger may become less well-aligned to the implant.

In some examples, the IMD may monitor the energy received during the recharge process. The IMD may communicate information representative of the energy received back to the recharging device, and the recharging device may use that information as at least part of an algorithm to determine if alignment should be adjusted. However, communication between the IMD and recharging device may not occur continuously during recharging. For example, energy transfer between the recharging device and IMD may disrupt communications between the devices, and energy transfer may be temporarily stopped in order to perform communication transfer. In the example of energy transfer using inductive coupling, the recharging using inductive coupling may interfere with inductive telemetry for communication. This interference may occur even if the frequencies for inductive energy transfer and inductive telemetry are different. Since communication transfer is only periodically provided, such as at a frequency of once every tens of seconds or even a minute or longer, the recharging device may not be able to quickly identify when alignment between the primary and secondary coils of the recharging device and IMD has been compromised and energy transfer has reduced in efficiency. The result can be longer recharge durations, wasted time for the patient, and increased IMD heating.

The devices, systems, and techniques of this disclosure may enable the recharging device to quickly determine, without communication with the medical device, when alignment with a medical device, such as an IMD, has changed and energy transfer may have been compromised. A recharging device can be configured to detect the presence of metal and a change to the metal that is present within the magnetic field of the recharge coil (e.g., a primary coil). For example, the recharging device can measure one or more electrical properties indicative of the loading on the recharge coil that occurs during the presence of metal. The metal that may be present that affects the loading on the recharge coil may include the secondary coil of the IMD, a metal housing of the IMD, or any other metal of the IMD. Although detecting metal with the recharge coil may not directly measure the actual power received by the IMD, the external recharging device may be able to provide quick feedback to the user that alignment should be improved when the loading from the metal changes (e.g., changes more than a predetermined percentage or amount) or is no longer detectable above a lower threshold amount. For example, the recharging device may measure loading several times a second. This loading detection frequency may be orders of magnitude greater than the communication frequency with the IMD.

In some examples, a recharging device may be configured to act like a metal detector while providing recharge energy through monitoring the load that is within a range of the recharge coil. The recharge device can measure electrical properties on the recharge coil and perform calculations based on one or more of those electrical properties. The recharging device may activate this metal detection feature in response to starting a recharge session, and the recharge device may not attempt to communicate with the IMD until the measured load is within a predetermined range. The recharge device may be configured to take an action in response to detecting that the load satisfies a certain threshold or take different actions in response to the load satisfying different respective thresholds. For example, the recharge device may attempt to communicate with the IMD in response to detecting a change in the load that exceeds a threshold. The communication may be used to attempt to retrieve coupling information from the IMD that indicates the actual power transfer to the IMD. If the IMD does not respond, or if the coupling information from the IMD confirms that coupling efficiency is not acceptable, the recharging device may present feedback to the user to move the recharging device to improve alignment and coupling efficiency. If the load has been reduced to a value indicative of the alignment no longer supporting energy transfer (e.g., the IMD is no longer within the usable magnetic field of the recharge coil), the recharging device can immediately present feedback to the user to move the recharge coil. In some examples, the recharging device may adjust one or more thresholds or the durations the load satisfies a threshold (e.g., the threshold may need to be exceeded for a predetermined amount of time). The system may also receive user input adjusting any of these times or thresholds. In some examples, increases or decreases in lead can indicate reduction in alignment and coupling efficiency, but other examples may only prompt feedback in response to reductions in loading of the recharge coil.

In some examples the recharging device may be configured to initiate communication with the IMD and/or present feedback to the user based on the load and another metric, such as an estimated charge current. This feedback may be used by the recharger to present a “speedometer” that indicates the estimated rate of energy transfer from the recharging device to the IMD. Such an implementation may be appropriate when the metal loading profile is symmetric with the IMD secondary coil such that loading changing may be proportional to coupling efficiency. For example, the estimated rate of energy transfer may be calculated based on the most recent current induced in the IMD battery and a ratio of the current loading to the last loading when the most recent current in the IMD battery was received via telemetry.

The systems, devices, and techniques described herein can provide various advantages. For example, an external recharging device that can detect changes in metal loading at a faster frequency than communication occurs with an IMD can improve feedback response time to inform the user to move the recharging device with respect to the IMD. In other words, the user may be able to move the recharging device (or recharge coil) in better alignment with the IMD secondary coil within seconds, in some examples, to reduce wasted charging time. In some examples, the recharging device may interrupt energy transfer to communicate with the IMD and receive updated coupling efficiency for providing accurate alignment feedback to the user. In any case, the examples herein provide for faster detection and user feedback when recharging alignment is reduced.

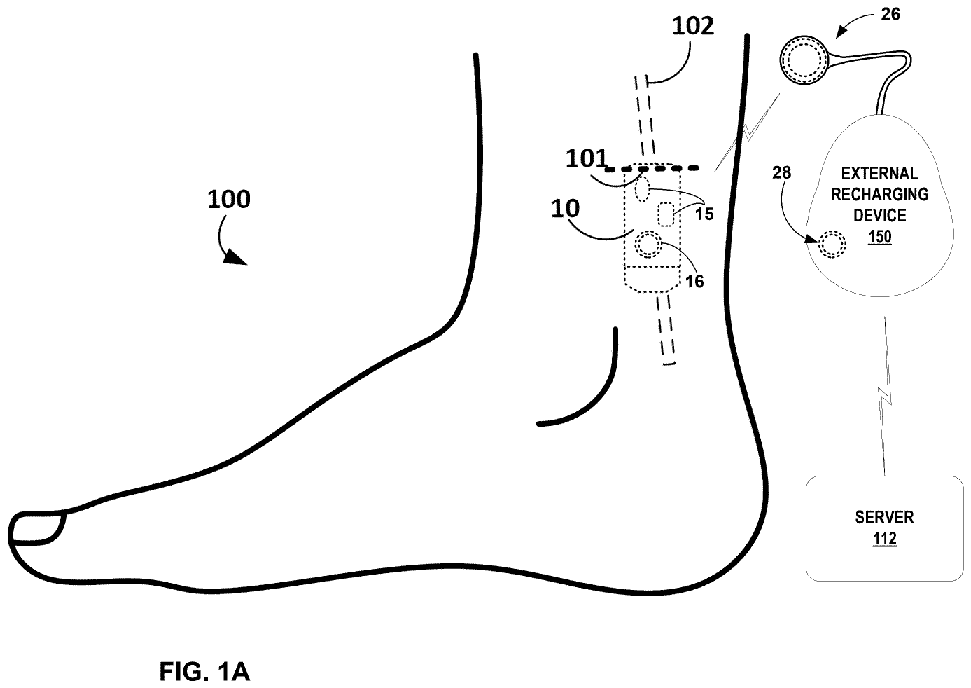

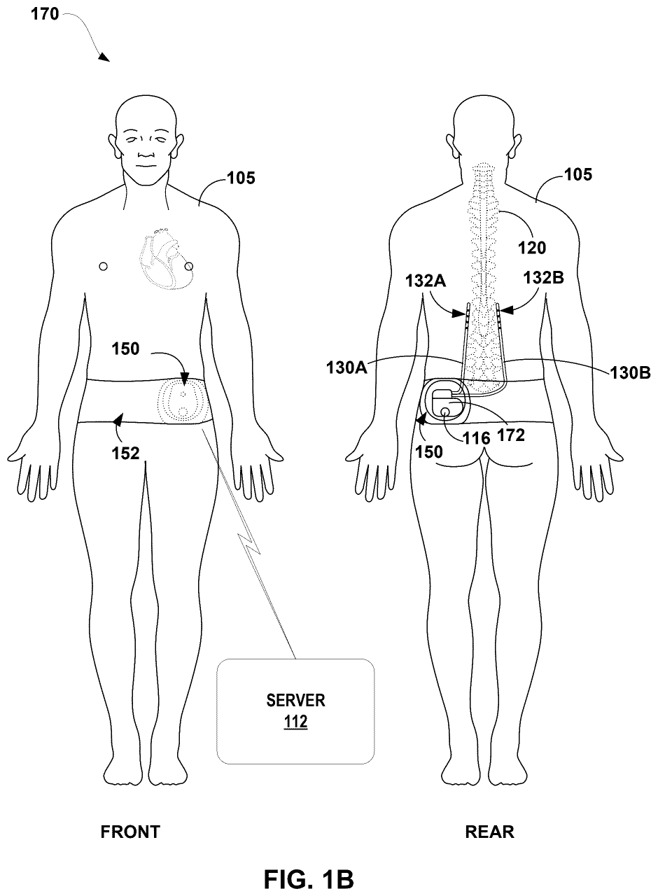

FIG. 1A is a conceptual diagram illustrating an example medical system 100 of this disclosure that includes an implantable medical device located near an ankle of a patient. The example of system 100 in FIG. 1A includes an implantable medical device 10, external computing device 150, and one or more servers 112. External computing device 150 may also be referred to as external recharging device 150 or recharger 150.

External computing device 150 includes one or more antenna, such as antenna 26 and antenna 28. In some examples, antenna 26 and/or antenna 28 may be coils configured to inductive energy transfer or inductive telemetry. Although coil 28 is shown in the side of external computing device 150, coil 28 may encompass much of the diameter of external computing device 15. In some examples, wherein antenna 26 and 28 perform different or similar functions, antennas 26 and 28 may be positioned as concentric coils within external computing device 150. In other examples, external computing device 150 may include three or more coils that are disposed at non-concentric positions that may or may not overlap with each other. These multiple coils may be configured to establish different magnetic fields that can provide a larger connection area and/or detect changes or movement of device 10 due to changes to loading to different coils of the multiple coils. External computing device 150 may be used to program or adjust settings of device 10 and may also recharge an electrical energy storage device, such as a battery, of device 10. External computing device 150 may also communicate with one or more servers 112. In other examples, a computing device separate from external computing device 150 (not shown in FIG. 1) may communicate with device 10 to adjust therapy and/or sensing parameters, download recorded data, and so on. In some examples, server 112 may be connected to, or represent, a remote computing device that may present a user interface to a user, such as a mobile computing device or remote desktop computer, etc. Any of these remote devices may interact with external computing device 150 or other devices via an internet connection and establish a cloud computing platform. In addition, system 100 may include additional devices, such as patient computing device (e.g., a device that may provide a patient user interface) or intermediary communication devices that may interact with each other using various communication protocols such as inductive telemetry and/or Bluetooth (e.g., normal Bluetooth of Bluetooth Low Energy (BLE) protocols).

The example of FIG. 1A is a side view of a patient's leg showing a leadless neurostimulation device 10 near the ankle adjacent to the tibial nerve 102. Device 10 can be implanted through the patient's skin and subcutaneous fat layer via a small incision 101 (e.g., about one to two cm or about 1.5 cm) above the tibial nerve on a medial aspect of the patient's ankle. While incision 101 is shown approximately horizontal to the length of the tibial nerve, other incisions or implantation techniques could be used according to physician preference. The example of FIG. 1A describes a neurostimulation implantable medical device for tibial nerve stimulation. In other examples, the techniques of this disclosure may apply to other rechargeable devices, such as implantable neurostimulation system for use in spinal cord stimulation therapy, deep brain stimulation, as well as to other types of medical devices without limitation. In this disclosure, device 10 may referred to as an implantable medical device (IMD) 10 or, in the example of a neurostimulation medical device, may be referred to as implantable neurostimulator (INS) 10.

Device 10 may be positioned adjacent to the region defined by flexor digitorum longus and soleus in which tibial nerve 102 is contained and implanted adjacent and proximal to a fascia layer. One or more electrodes of device 10 may face toward tibial nerve 102. Other electrodes, e.g., electrodes 15 may be located in other positions on device 10. Though not shown in FIG. 1A, device 10 may also connect to one or more leads comprising one or more electrodes (not shown in FIG. 1A).

Device 10 may be constructed of any polymer, metal, or composite material sufficient to house the components of device 10. In this example, device 10 may be constructed with a biocompatible housing, such as titanium or stainless steel, or a polymeric material such as silicone, polyurethane, or polysulfone, and surgically implanted at a site in patient near the tibial nerve, in some examples, while in other examples, implanted near the pelvis, abdomen, or buttocks. The housing of device 10 may be configured to provide a hermetic seal for components, such as a rechargeable power source. In addition, the housing of device 10 may be selected of a material that facilitates receiving energy to charge the rechargeable power source.

Testing of neurostimulation device 10 may be performed to determine if device 10 has been properly positioned in proximity to tibial nerve 102 to elicit a desired response from an applied electrical stimulation. In an example, device 10 is controlled by an external programmer to deliver test stimulation, and one or more indicative responses are monitored, such as toe flexion from simulation of the tibial motor neurons controlling the flexor hallucis brevis or flexor digitorum brevis, or a tingling sensation in the heel or sole of the foot excluding the medial arch. If such testing does not elicit appropriate motor or sensory responses, the practitioner may reposition device 10 and retest.

Once a practitioner has determined device 10 is properly positioned to provide an appropriate patient response to delivered stimulation therapy, the housing of device can be secured in place if needed. Such anchoring means may be optional as the natural shape of the region in which device 10 is implanted, and the shape of device 10 itself may have good compatibility with the surrounding tissue thus preventing device 10 from shifting or rolling after implantation. In some examples, leadless neurostimulation device 10 may further include one or more suture points to help secure device 10 to fascia or other parts of the patient. In some examples, a suture anchor may be included, such as at the distal end of the housing of device 10. In contrast to other approaches, leadless neurostimulation device 10 may not require the patient's fascia layer near the implant site to be disturbed which may reduce risks affiliated with alternative procedures. Further, device 10 is a unitary structure and may be hermetically sealed.

During normal operation after implantation, an electrical stimulation signal may be transmitted between one or more electrodes through the fascia layer. The electrical signal may be used to stimulate tibial nerve 102 which may be useful in the treatment of overactive bladder (OAB) symptoms of urinary urgency, urinary frequency and/or urge incontinence, or fecal incontinence.

One type of therapy for treating bladder dysfunction includes delivery of electrical stimulation to a target tissue site within a patient to cause a therapeutic effect during delivery of the electrical stimulation. For example, delivery of electrical stimulation from device 10 to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, brain, or branches of any of the aforementioned nerves, may provide a therapeutic effect for bladder dysfunction, such as a desired reduction in frequency of bladder contractions. In some cases, electrical stimulation of the tibial nerve may modulate afferent nerve activities to restore urinary function or large intestine function.

In the example of a rechargeable power source, the rechargeable power source of device 10 may include one or more capacitors, batteries, or other components (e.g., chemical, or electrical energy storage devices). Example batteries may include lithium-based batteries, nickel metal-hydride batteries, or other materials. The rechargeable power source may be replenished, refilled, or otherwise capable of increasing the amount of energy stored after energy has been depleted. The energy received from secondary coil 16 may be conditioned and/or transformed by a charging circuit (e.g., via inductive energy transfer). The charging circuit may then send an electrical signal used to charge the rechargeable power source when the power source is fully depleted or only partially depleted.

External computing device 150 may be used to recharge the rechargeable power source within device 10 implanted in the patient. External computing device 150 may be a hand-held device, a portable device, or a stationary charging system. External computing device 150 may include components necessary to charge device 10 through tissue of the patient. External computing device 150 may include an internal energy transfer coil 28 or external energy transfer coil 26, also referred to as primary coil 26 or primary coil 28. In other examples, external computing device may only include internal primary coil 28 and omit the use of external primary coil 26. External computing device 150 may be referred to as a recharging device in some examples because it is configured to transfer energy to, and recharge, device 10.

External computing device 150 may include a housing to enclose operational components such as a processor, memory, user interface, telemetry circuitry, power source, and charging circuit configured to transmit energy to secondary coil 16 via energy transfer coil 26 and/or 28. Although a user may control the recharging process with a user interface of external computing device 150, external computing device 150 may alternatively be controlled by another device, e.g., an external programmer, a computing device of servers 112, where such servers may include a tablet computer, laptop or other similar computing device. External computing device 150, and any computing device of servers 112 may include a touch-screen user interface. In other examples, external computing device 150 may be integrated with an external programmer, such as a patient programmer carried by the patient.

External computing device 150 and device 10 may utilize any wireless power transfer techniques that are capable of recharging the power source of device 10 when device 10 is implanted within the patient. In some examples, system 100 may utilize inductive coupling between primary coils (e.g., energy transfer coil 28) and secondary coils (e.g., secondary coil 16) of external computing device 150 and device 10. In inductive coupling, energy transfer coil 28 is placed near implanted device 10 such that energy transfer coil 28 is aligned with secondary coil 16 of device 10. External computing device 150 may then generate an electrical current in energy transfer coil 28 based on a selected power level for charging the rechargeable power source of device 10. When the primary and secondary coils are aligned, or partially aligned, the electrical current in the primary coil may magnetically induce an electrical current in secondary coil 16 within device 10. When the primary and secondary coils are not fully aligned, the energy transfer efficiency is reduced. In response to the energy transfer being reduced below a threshold, external computing device 150 may provide feedback to the user in the form of an audible, visual, and/or tactile alert to move the primary coil with respect to the secondary coil to improve alignment and coupling efficiency. Since the secondary coil is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between external computing device 150 and device 10.

The degree (or quality) of alignment of a primary coil (either coil 26 or 28) with secondary coil 16 may affect the efficiency of the energy transfer between external computing device 150 and device 10. Energy transfer efficiency may be calculated in several ways, such a ratio between the amount of power produced by the primary coil and the amount of power received by the secondary coil. In some examples, an efficient energy transfer alignment may be when the primary coil, e.g., coil 28 is concentric with secondary coil 16. In other examples, an energy efficient transfer alignment may be when the primary coil center is offset from the center of the secondary coil. In any event, the components of system 100 may be configured to provide feedback to a patient to help spatially align the primary and secondary coils that enable an efficient transfer alignment. In less efficient alignments, the energy produced by the primary coil 28 results in less current induced in secondary coil 16. For example, processing circuitry within device 10 (not shown in FIG. 1) may receive an indication of a quality of alignment with power transmitting device, e.g., a primary coil of external computing device 150. As noted above, the indication of the quality of alignment may include any of several system metrics to determine the quality of alignment, such as power transfer efficiency, the magnitude of current induced in device 10, a measure of heating within device 10, or any other indication of energy transfer efficiency and/or alignment. Responsive to the indication of the quality of alignment, the processing circuitry of device 10 may cause the stimulation generation circuitry of device 10 (not shown in FIG. 1) to deliver haptic stimulation representative of the quality of alignment. External computing device 150 may also be configured to determine the loading of the primary coil which may be indicative of a change to orientation of secondary coil 16 with respect to the primary coil in external computer device 150. External computing device 150 may the be able to provide feedback more quickly to the user to improve alignment even if external computer device 150 does not have updated alignment information from device 10.

The feedback to the user may be in the form of an audible alert, such as one or more “beeps” that the user can interpret as a request to move the primary coil back in alignment with the secondary coil of device 10. In some examples, the feedback may be in the form of a user interface display that provides light, graphical, or textual information to the user regarding the reduction in coupling efficiency and/or need to improve alignment of the primary and secondary coils. In some examples, external computing device 150 may provide tactile (e.g., haptic) feedback in the form of a vibration or other movement that the user can detect as an alert that alignment needs to be improved for coupling.

Energy transfer coil 26 and 28 may include a wound wire (e.g., a coil) (not shown in FIG. 1A). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Energy transfer coil 28 may be external of the housing of external computing device 150 such that energy transfer coil 28 can be placed on the skin of the patient proximal to device 10. In some examples, energy transfer coil 28 may be disposed on the outside of the housing or even within a separate housing.

Either primary coil 26 and/or 28 of system 100 may include a heat sink device (not shown in FIG. 1A). In the example of system 100, external computing device 150 is the power transmitting unit and device 10 is the power receiving unit. device 10 may be in a flipped or non-flipped position.

As noted above, in this disclosure external computing device 150 may also be referred to as recharger 150. External computing device 150 may include a user interface to receive control inputs from a user, such as the patient, medical professional, or other caregiver. The user interface of external computing device 150 may also provide information to a user, including the quality of alignment, whether device 10 is ON and delivering therapy, whether external computing device 150 is wirelessly communicating with device 10 and so on. In some examples, the user interface may provide directional feedback indicating to the user which direction to move external computing device 150 to improve alignment.

In some examples, external computing device 150 may receive wireless communication from device 10 that include the amount of power delivered to the electrical energy storage device of device 10, which may be referred to as closed loop charging. In other words, system 100 may measure efficiency, such as IMD efficiency, to determine whether the relative position of primary coil 26 and secondary coil 16 may be in a less desirable relative position. As described herein, external computing device 150 may use this information from device 10 when possible, but external computing device 150 may determine metal loading from the primary coil and use this loading on the primary coil to provide feedback on changes to the alignment when the device 10 information is not available.

A variety of system metrics are available to external computing device 150 from computations of power and heat and from metrics communicated to the recharger from IMD 14. Processing circuitry of system 100, e.g., processing circuitry of external computing device 150, processing circuitry of servers 112, and/or processing circuitry of device 10, may calculate any of the values described herein. These metrics may include but are not limited to: battery current (lins_batt), Power Transfer Efficiency (Pins_batt/Ptank), IMD Efficiency (Pins_batt/Qins) or (Pins_batt/Pins). Pins_batt may be the power delivered to the IMD batters, Ptank may be the power delivered from the tank circuit of the primary coil, Pins may be the power received by the IMD secondary coil, and Qins may be the reactive power received at the IMD. Analysis of system characterization data that the IMD efficiency, which may be measured by device 10 and communicated to external computing device 150, may be an example indicator of when the recharger primary coil 26 is concentric with secondary coil 16. A concentric relative position of primary coil 26 and secondary coil 16 may be in positions with the lowest overall transient thermal response (increase in temp for the same heat). In some examples, if the IMD is asymmetrical, the energy transfer in concentric positions (e.g., near 0, −20 in X and Y) may be higher and the battery of IMD 14 may charge faster.

Therefore, there may be an exponential relationship or linear between the IMD efficiency (or Qins or Pins), which may also be referred to as INS efficiency in this disclosure, and the overall thermal dose in units of CEM 43 (i.e., an equivalent time at 43 degrees Celsius). The power transfer efficiency on the other hand may be more skewed towards the geometrical center of the device 10 (near 0, 0 in X and Y). In some examples both power transfer efficiency and IMD efficiency metrics may be lower when primary coil 26 is positioned over the header of device 10, which may lead to decreased efficiency and a less desirable thermal profile (e.g., an increase in temperature for the same heat). The header or case of device 10 may be non-metallic (e.g., polysulfone or ceramic) and contain connections for one or more leads connected to electrodes or other sensors. Furthermore, at such positions the time to charge may be longer so the overall thermal dose could be higher than quicker charging periods that result from more efficient coupling. In some examples, processing circuitry of external computing device 150 may determine the impedance of primary coil 28, or coil 26, to calculate an estimate for the amount of heating of the power receiving unit, e.g., device 10. The cost of suboptimal alignment is that recharging make take longer and may generate more heat, e.g., in device 10 and/or the surrounding tissue, than is strictly necessary. In this manner, the techniques of this disclosure may provide advantages over other subcutaneous wireless power transfer techniques by establishing, and maintaining, primary to secondary coil alignment. In addition, external computing device 150 may be configured to detect load changes on the primary coil from one or more electrical parameters of the coil and, responsive to the load (or the change in the load) satisfying one or more thresholds, present feedback to the user and/or request information from device 10 to attempt urgent improvements to the alignment of the primary and secondary coils.

In some examples described herein, an external charging device, such as external computing device 150, may include a recharge coil (e.g., a primary coil) configured to transfer energy to an IMD (e.g., device 10) (which may include a secondary coil) and detect metal loading, charging circuitry coupled to the recharge coil and configured to determine one or more electrical properties of the recharge coil during the transfer of energy, and processing circuitry. The processing circuitry may be configured to determine, based on the one or more electrical properties, a load on the recharge coil, compare the load on the recharge coil to one or more thresholds, and responsive to the load satisfying the threshold, perform an action associated with the transfer of energy to the IMD.

The processing circuitry may be configured to determine, based on the comparison of the load to the one or more thresholds, that the load has dropped below the one or more thresholds which indicates that the recharge coil is insufficiently aligned to a secondary coil of the IMD. The action may include controlling a user interface to display a representation indicative of improper coupling between the recharge coil and the secondary coil. Instead of, or in addition to a display, the external charging device may be configured to present audible “beeps” or other sounds (e.g., verbal instructions) to the user to request the user improve the alignment of the primary and secondary coils. Tactile feedback may also, or alternatively, be used in other examples.

In some examples, the action performed in response to the load satisfying a threshold may include initiating communication with the IMD. The processing circuitry may be configured to, responsive to initiating communication with the IMD, receive coupling information from the IMD indicative of coupling of the recharge coil to a secondary coil of the IMD. In this manner, the communication transmitted to the IMD may be a request for an updated power transfer value or other coupling efficiency metric. The processing circuitry may also be configured to, responsive to communication initiation with the IMD, control the charging circuitry to charge for a period of time, wherein the coupling information is representative of coupling efficiency for the period of time. This charging performed may be used so that the IMD can calculate the coupling information during the charging. This charging session may be relatively short, such as only a few seconds in some examples.

Responsive to determining that no communication has been initiated between the external charging device and the IMD, control a user interface to present an indication to a user that coupling with the IMD is below a threshold. In some examples, the processing circuitry may be configured to control a user interface to present an indication to a user that coupling with the IMD is below a threshold. The processing circuitry may be configured to estimate a recharge coupling efficiency based on the load on the recharge coil. This recharge coupling efficiency may be calculated with the load value and the most recent charging current to the IMD as inputs. In some examples, the processing circuitry may calculate the estimated coupling efficiency using a ratio of the latest load to the load associated with the most recent charging current to the IMD. That ratio can then be multiplied by the most recent charging current. In this manner, the processing circuitry may estimate the recharge coupling efficiency based on the load at the primary coil, without real time communication from the IMD. The processing circuitry may also be configured to control a user interface to display a representation indicative of the estimated recharge coupling efficiency. This representation may be a “speedometer” or other graphical, numerical, or textual representation of the estimated recharge coupling efficiency. In some examples, the external recharge device may use this estimated recharge coupling efficiency to update the representation of the “speedometer” to the user at all times, even when communication is not being received from the IMD. The external recharging device may then “correct” the user interface and coupling efficiency in response to receiving updated communication and power transfer values from the IMD. In some examples, the user interface may provide an estimated time to “full” (e.g., 25 minutes remaining) to indicate the remaining charging for the device.

FIG. 1B is a conceptual diagram illustrating an example system 170 that includes an IMD 172 configured to deliver spinal cord stimulation (SCS) therapy and an external computing device 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. M ore particularly, the disclosure will refer to an implantable SCS system for purposes of illustration, but without limitation as to other types of medical devices or other therapeutic applications of medical devices. In the example of FIG. 1B, system 170 includes IMD 172 with antenna 116, external computing device 150 and servers 112, which are, respectively examples of IMD 110 with antenna 16, external computing device 150 and servers 112 described above in relation to FIG. 1A and may have the same or similar functions and characteristics. As discussed above, service 112 may include or be connected to other computing devices that may be configured to provide a user interface for interacting with other devices such as external computing device 150 and/or IMD 172. Although antenna 116 is shown as using a small volume of IMD 172, antenna 116 may be disposed in as large a space as possible, such as around a majority of the housing.

As shown in FIG. 1B, system 170 includes an IMD 172, leads 130A and 130B, and external computing device 150 shown in conjunction with a patient 105, who is ordinarily a human patient. In the example of FIG. 1B, IMD 172 is an implantable electrical stimulator that is configured to generate and deliver electrical stimulation therapy to patient 105 via one or more electrodes of electrodes 132A and 132B, respectively on leads 130A and/or 130B (collectively, “leads 130”), e.g., for relief of chronic pain or other symptoms. In other examples, IMD 172 may be coupled to a single lead carrying multiple electrodes or more than two leads each carrying multiple electrodes. IMD 172 may include an electrical connector configured to connect to the electrical leads, e.g., in the header of IMD 172. In other examples, IMD 172 may include electrodes in contact with patient tissue on the device and not connected through leads 130, similar to IMD 10 described above in relation to FIG. 1A.

IMD 172 may be a chronic electrical stimulator that remains implanted within patient 105 for weeks, months, or even years. In other examples, IMD 172 may be a temporary, or trial, stimulator used to screen or evaluate the efficacy of electrical stimulation for chronic therapy. In one example, IMD 172 is implanted within patient 105, while in another example, IMD 172 is an external device coupled to percutaneously implanted leads. In some examples, the stimulation signals, or pulses, may be configured to elicit detectable ECAP signals that IMD 172 may use to determine the posture state occupied by patient 105 and/or determine how to adjust one or more parameters that define stimulation therapy.

The techniques of this disclosure may also apply to other devices, including wearable devices, that may be located elsewhere on patient 105. Some examples may include devices located near the head or pectoral muscle for DBS, near the tibial region as in the example of FIG. 1A, near the heart for cardiac therapy and/or monitoring, and so on.

In other words, although in one example IMD 172 takes the form of an SCS device, in other examples, IMD 172 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine parameters that affect stimulation thresholds (e.g., perception thresholds and detection thresholds) associated any one of the aforementioned IMDs and then use a stimulation threshold to inform the intensity (e.g., stimulation levels) of therapy. For example, changing stimulation parameters such as the number of pulses in a burst, the number of bursts over a duration, the pulse width of a pulse in a burst, the ON-time, the OFF-time, a pattern of pulses over a duration and other parameters may change the intensity as well as the efficacy of the therapy to relieve the symptoms.

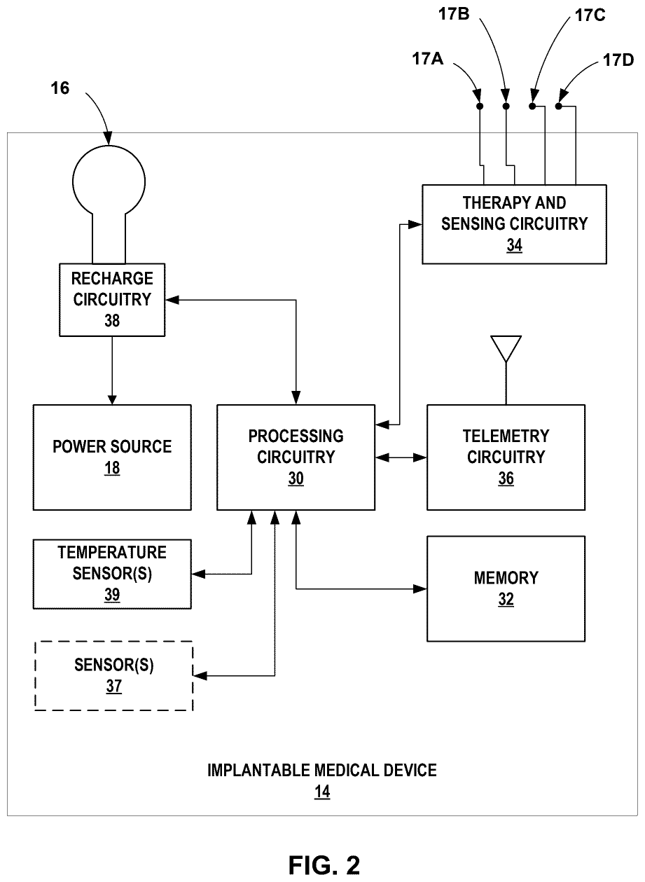

As with IMD 110 described above in relation to FIG. 1A, IMD 172 may be constructed of any polymer, metal, ceramic, or composite material sufficient to house the components of IMD 172 (e.g., components illustrated in FIG. 2) within patient 105. In this example, IMD 172 may be constructed with a biocompatible housing, such as titanium or stainless steel, or a polymeric material such as silicone, polyurethane, polysulfone, or a liquid crystal polymer, and surgically implanted at a site in patient 105 near the pelvis, abdomen, or buttocks. In other examples, IMD 172 may be implanted within other suitable sites within patient 105, which may depend, for example, on the target site within patient 105 for the delivery of electrical stimulation therapy. The outer housing of IMD 172 may be configured to provide a hermetic seal for components, such as a rechargeable or non-rechargeable power source. In addition, in some examples, the outer housing of IMD 172 is selected from a material that facilitates receiving energy to charge the rechargeable power source.

Electrical stimulation energy, which may be constant current or constant voltage pulses, for example, is delivered from IMD 172 to one or more target tissue sites of patient 105 via one or more electrodes 132A and 132B (collectively electrodes 132) of implantable leads 130. In the example of FIG. 1B, leads 130 carry electrodes that are placed adjacent to the target tissue of spinal cord 120. One or more of electrodes 132 may be disposed at a distal tip of a lead 130 and/or at other positions at intermediate points along the lead. Leads 130 may be implanted and coupled to IMD 172. Electrodes 132 may transfer electrical stimulation generated by an electrical stimulation generator in IMD 172 to tissue of patient 105. Electrodes 132 may also sense bioelectrical signals of patient 105.

Although leads 130 may each be a single lead, lead 130 may include a lead extension or other segments that may aid in implantation or positioning of lead 130. In some other examples, IMD 172 may be a leadless stimulator with one or more arrays of electrodes arranged on a housing of the stimulator rather than leads that extend from the housing, as shown in IMD 110 of FIG. 1A. In addition, in some other examples, system 170 may include one lead or more than two leads, each coupled to IMD 172 and directed to similar or different target tissue sites.

Electrodes 132A and 132B of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes 132A and 132B via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 172 through the electrodes of leads 130 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 170 based on one or more factors or user input.

Similarly, sensing bioelectrical signals may use a variety of combinations of electrodes on leads 130, the housing of IMD 172, or other sensors connected directly or indirectly to IMD 172. In some examples, lead 130 includes one or more sensors configured to allow IMD 172 to monitor one or more parameters of patient 105, such as patient activity, pressure, temperature, or other characteristics. The one or more sensors may be provided in addition to, or in place of, therapy delivery by lead 130.

Although FIG. 1B is directed to SCS therapy, e.g., used to treat pain, in other examples system 170 may be configured to treat any other condition that may benefit from electrical stimulation therapy. For example, system 170 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 170 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105. In other examples, IMD 172 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, drug pumps and so on.

IMD 172 is configured to deliver electrical stimulation therapy to patient 105 via selected combinations of electrodes carried by one or both of leads 130, alone or in combination with an electrode carried by or defined by an outer housing of IMD 172. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 1B, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

IMD 172 is configured to generate and deliver electrical stimulation therapy to a target stimulation site within patient 105 via the electrodes of leads 130 to patient 105 according to one or more therapy stimulation programs. A therapy stimulation program defines values for one or more parameters (e.g., a parameter set) that define an aspect of the therapy delivered by IMD 172 according to that program. For example, a therapy stimulation program that controls delivery of stimulation by IMD 172 in the form of pulses may define values for voltage or current pulse amplitude, pulse width, pulse rate (e.g., pulse frequency), electrode combination, pulse shape, etc. for stimulation pulses delivered by IMD 172 according to that program. In some examples, parameters may include sequences of pulses, for example a “burst” of pulses with gradually increasing current magnitudes, or some other sequence. In some examples, IMD 172 may deliver therapy for a given duration and stop delivering therapy for a given duration. In other words, parameters of the electrical stimulation therapy may include an ON-time and an OFF-time. In some examples, an ON-time may be a few seconds or minutes and the OFF-time may also be for a few seconds or minutes. The ON-time may be equal to the OFF-time in some examples, while in other examples the ON-time and the OFF-time may be unequal durations (e.g., the ON-time may only be for 30 minutes one, two, or three days a week with the remaining times being OFF-time).

In the example of FIG. 1B, external computing device 150 may be placed near IMD 172 to communicate and/or transfer power to IMD 172. In some examples, external computing device 150 may be held in place by a belt or straps 152. In some examples belt 152 may include a pouch that accepts external computing device 150. For implant locations on the hip, as shown in FIG. 1B, patient 105 may find it difficult to position the primary coil of external computing device 150 and to maintain the relative position of the primary and secondary coils. The feedback recharging alignment of this disclosure may provide quick feedback to patient 105, based on the loading detected in the primary coil, to adjust the position of external computing device 150. Examples of the user interface may include indicator lights, audio feedback, or graphics displayed on a graphical user interface (GUI) such as a tablet computer, smart phone and so on of servers 112.

As described above in relation to FIG. 1A, processing circuitry of system 170 may determine the quality of alignment based on any combination of system metrics. As noted above, some examples of system metrics may include a power reception efficiency, a power transfer efficiency, electrical current magnitude, calculated amount of heat, and so on. In some examples system metrics may include an indication of a metal detection magnitude, e.g., based on the proximity of the primary coil to the housing of IMD 172 and/or metal inside of the housing such as secondary coil 116.

In some examples, a user, such as a clinician or patient 105, may interact with a user interface of an external computing device, such as external computing device 150 or server 112, to program IMD 172. In some examples, external computing device 150 may also be referred to as a programmer. Programming of IMD 172 may refer generally to the generation and transfer of commands, programs, or other information to control the operation of IMD 172. In this manner, IMD 172 may receive the transferred commands and programs from external computing device 150 to control stimulation, such as electrical stimulation therapy (e.g., informed pulses), control stimulation (e.g., control pulses), haptic stimulation, sensing and so on. For example, external computing device 150 may transmit therapy stimulation programs, ECAP test stimulation programs, stimulation parameter adjustments, therapy stimulation program selections, ECAP test program selections, user input, or other information to control the operation of IMD 172, e.g., by wireless telemetry or wired connection.

As described above in relation to FIG. 1A, information may be transmitted between external computing device 150 and IMD 172. Therefore, IMD 172 and external computing device 150 may communicate via wireless communication using any techniques known in the art. Examples of communication techniques may include, for example, radiofrequency (RF) telemetry and inductive coupling, but other techniques are also contemplated. In some examples, external computing device 150 includes a communication head, e.g., antenna 26 depicted in FIG. 1A, that may be placed proximate to the patient's body near the IMD 172 implant site to improve the quality or security of communication between IMD 172 and external computing device 150. Communication between external computing device 150 and IMD 172 may occur during power transmission or separate from power transmission. Although antenna 26 is shown as separate from antenna 28, antenna 26 may be concentric with antenna 28 in other examples (and both antennas may be within the housing).

In the example of FIG. 1B, IMD 172 described as performing a plurality of processing and computing functions. However, external computing device 150 and/or servers 112 instead may perform one, several, or all of these functions. In this alternative example, IMD 172 functions to relay sensed signals to external computing device 150 for analysis, and external computing device 150 transmits instructions to IMD 172 to adjust the one or more parameters defining the electrical stimulation therapy based on analysis of the sensed signals. For example, IMD 172 may relay the sensed signal indicative of an ECAP to external computing device 150. External computing device 150 may compare the parameter value of the ECAP to the target ECAP characteristic value, and in response to the comparison, external computing device 150 may instruct IMD 172 to adjust one or more stimulation parameter that defines the electrical stimulation informed pulses and, in some examples, control pulses, delivered to patient 105.

FIG. 2 is a block diagram illustrating example components of the medical device of FIG. 1A. Implantable medical device 14 is an example of device 10 described above in relation to FIG. 1A and IMD 172 of FIG. 2 and may have the same or similar functions and characteristics. In the example illustrated in FIG. 2, IMD 14 includes temperature sensor 39, coil 16, processing circuitry 30, therapy and sensing circuitry 34, recharge circuitry 38, memory 32, telemetry circuitry 36, power source 18, and one or more sensors 37, such as an accelerometer. In other examples, IMD 14 may include a greater or a fewer number of components, e.g., in some examples, IMD 14 may not include temperature sensor 39 or sensors 37. In general, IMD 14 may comprise any suitable arrangement of hardware, alone or in combination with software and/or firmware, to perform the various techniques described herein attributed to IMD 14 and processing circuitry 30, and any equivalents thereof.

Processing circuitry 30 of IMD 14 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 14 may include a memory 32, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 30 to perform the actions attributed to this circuitry. Moreover, although processing circuitry 30, therapy and sensing circuitry 34, recharge circuitry 38, telemetry circuitry 36, and temperature sensor 39 are described as separate modules, in some examples, some combination of processing circuitry 30, therapy and sensing circuitry 34, recharge circuitry 38, telemetry circuitry 36 and temperature sensor 39 are functionally integrated. In some examples, processing circuitry 30, therapy and sensing circuitry 34, recharge circuitry 38, telemetry circuitry 36, and temperature sensor 39 correspond to individual hardware units, such as ASICs, DSPs, FPGAs, or other hardware units. For example, components of IMD 14 may be implemented as separate circuits in some examples. In other examples, two or more components of IMD 14 may be implemented on a single integrated circuit, e.g., including processing circuitry 30, telemetry circuitry 36, memory 32, therapy and sensing circuitry 34, and so on. In this disclosure, therapy, and sensing circuitry 34 may be referred to as therapy circuitry 34, for simplicity.

Memory 32 may store therapy programs or other instructions that specify therapy parameter values for the therapy provided by therapy circuitry 34 and IMD 14. In some examples, memory 32 may also store temperature data from temperature sensor 39, instructions for recharging rechargeable power source 18, thresholds, instructions for communication between IMD 14 and an external computing device, or any other instructions required to perform tasks attributed to IMD 14. Memory 32 may be configured to store instructions for communication with and/or controlling one or more temperature sensors of temperature sensor 39. In various examples, memory 32 stores information related to determining the temperature of housing 19 and/or exterior surface(s) of housing 19 of IMD 14 based on temperatures sensed by one or more temperature sensors, such as temperature sensor 39, located within IMD 14.

For example, memory 32 may store programming settings such as parameters for electrical stimulation therapy output, e.g., magnitude, pulse width, and so on. Memory 32 may store parameters and other settings, such as communication with an external charging device (e.g., device 150). Settings may be individualized based on patient preference and/or patient physiology.

Instructions stored at memory 32 when executed by processing circuitry 30 may determine whether a sensed bioelectrical signal is valid, such as and ECAP or other signal in response to an output electrical stimulation therapy event. Memory 32 may store programming instructions that when executed by processing circuitry 30 cause processing circuitry 30 to cause electrical stimulation circuitry therapy circuitry 34 to deliver electrical stimulation therapy to a target nerve of a patient.

Therapy and sensing circuitry 34 may generate and deliver electrical stimulation under the control of processing circuitry 30. Therapy and sensing circuitry 34 may also output non-therapy stimulation, such as control pulses and haptic stimulation. In some examples, processing circuitry 30 controls therapy circuitry 34 by accessing memory 32 to selectively access and load at least one of the stimulation programs to therapy circuitry 34. For example, in operation, processing circuitry 30 may access memory 32 to load one of the stimulation programs to therapy circuitry 34. In such examples, relevant stimulation parameters may include a voltage amplitude, a current amplitude, a pulse rate, a pulse width, a duty cycle, or the combination of electrodes 17A, 17B, 17C, and 17D (collectively “electrodes 17”) that therapy circuitry 34 may use to deliver the electrical stimulation signal as well as sense biological signals. In other examples, IMD 14 may have more or fewer electrodes than the four shown in the example of FIG. 2. In some examples electrodes 17 may be part of or attached to a housing of IMD 14, e.g., a leadless electrode. In other examples, one or more of electrodes 17 may be part of a lead implanted in or attached to a patient to sense biological signals and/or deliver electrical stimulation, as described above in relation to FIG. 1A.

In some examples, one or more electrodes connected to therapy circuitry 34 may connect to one or more sensing electrodes, e.g., attached to housing of IMD 14. In some examples the electrodes may be configured to detect an evoked motor response caused by the electrical stimulation therapy event, or other bioelectrical signals such as ECAPs, impedance and so on.

IMD 14 also includes components to receive power to recharge rechargeable power source 18 when rechargeable power source 18 has been at least partially depleted. As shown in FIG. 2, IMD 14 includes coil 16 and recharge circuitry 38 coupled to rechargeable power source 18. Recharge circuitry 38 may be configured to charge rechargeable power source 18 with the selected power level determined by either processing circuitry 30 or an external charging device, such as external computing device 150 described above in relation to FIG. 1A. Recharge circuitry 38 may include any of a variety of charging and/or control circuitry configured to process or convert current induced in coil 16 into charging current to charge power source 18. For example, recharge circuitry 38 may include measurement circuitry configured to determine a magnitude of current received by secondary coil 16, a magnitude of current delivered to power source 18, and other measurements. Recharge circuitry 38 may send such measurements to processing circuitry 30 to be used in system metrics, e.g., to determine a quality of alignment.

Secondary coil 16 may include a coil of wire or other device capable of inductive coupling with a primary coil disposed external to patient 12. Although secondary coil 16 is illustrated as a simple loop of in FIG. 2, secondary coil 16 may include multiple turns of conductive wire. Secondary coil 16 may include a winding of wire configured such that an electrical current can be induced within secondary coil 16 from a magnetic field. The induced electrical current may then be used to recharge rechargeable power source 18.

Recharge circuitry 38 may include one or more circuits that process, filter, convert and/or transform the electrical signal induced in the secondary coil to an electrical signal capable of recharging rechargeable power source 18. For example, in alternating current induction, recharge circuitry 38 may include a half-wave rectifier circuit and/or a full-wave rectifier circuit configured to convert alternating current from the induction to a direct current for rechargeable power source 18. The full-wave rectifier circuit may be more efficient at converting the induced energy for rechargeable power source 18 when coupling is good. However, a half-wave rectifier circuit may be used to store energy in rechargeable power source 18 at a slower rate when coupling is good or faster when coupling is less optimal. In some examples, recharge circuitry 38 may include both a full-wave rectifier circuit and a half-wave rectifier circuit such that recharge circuitry 38 may switch between each circuit to control the charging rate of rechargeable power source 18 and temperature of IMD 14.

Rechargeable power source 18 may include one or more capacitors, batteries, and/or other energy storage devices. Rechargeable power source 18 may deliver operating power to the components of IMD 14. In some examples, rechargeable power source 18 may include a power generation circuit to produce the operating power. Rechargeable power source 18 may be configured to operate through many discharge and recharge cycles. Rechargeable power source 18 may also be configured to provide operational power to IMD 14 during the recharge process. In some examples, rechargeable power source 18 may be constructed with materials to reduce the amount of heat generated during charging. In other examples, IMD 14 may be constructed of materials and/or using structures that may help dissipate generated heat at rechargeable power source 18, recharge circuitry 38, and/or secondary coil 16 over a larger surface area of the housing of IMD 14.

Although rechargeable power source 18, recharge circuitry 38, and secondary coil 16 are shown as contained within the housing of IMD 14, in alternative implementations, at least one of these components may be disposed outside of the housing. For example, in some implementations, secondary coil 16 may be disposed outside of the housing of IMD 14 to facilitate better coupling between secondary coil 16 and the primary coil of external charging device. In other examples, power source 18 may be a primary power cell and IMD 14 may not include recharge circuitry 38 and recharge coil 16.

Processing circuitry 30 may also control the exchange of information with an external computing device using telemetry circuitry 36. In the example of FIG. 2, telemetry circuitry 36 may be configured for wireless communication using radio frequency protocols, such as BLUETOOTH, or similar RF protocols, as well as using inductive communication protocols (e.g., inductive telemetry). Telemetry circuitry 36 may include one or more antennas configured to communicate with external charging device, for example. Processing circuitry 30 may transmit operational information and receive therapy programs or therapy parameter adjustments via telemetry circuitry 36. Also, in some examples, IMD 14 may communicate with other implanted devices, such as stimulators, control devices, or sensors, via telemetry circuitry 36. In addition, telemetry circuitry 36 may be configured to control the exchange of information related to sensed and/or determined temperature data, for example temperatures sensed by and/or determined from temperatures sensed using temperature sensor(s) 39. For example, temperature sensors may be used to determine the location of the charger with respect to the IMD in a plane. In some examples, telemetry circuitry 36 may communicate using inductive communication, and in other examples, telemetry circuitry 36 may communicate using RF frequencies separate from the frequencies used for inductive charging.

In some examples, processing circuitry 30 may transmit additional information to external charging device related to the operation of rechargeable power source 18. For example, processing circuitry 30 may use telemetry circuitry 36 to transmit indications that rechargeable power source 18 is completely charged, rechargeable power source 18 is fully discharged, the amount of charging current output by recharge circuitry 38 e.g., to power source 18, or any other charge status of rechargeable power source 18. In some examples, processing circuitry 30 may use telemetry circuitry 36 to transmit instructions to external charging device, including instructions regarding further control of the charging session, for example instructions to lower the power level or to terminate the charging session, based on the determined temperature of the housing/external surface 19 of the IMD.

Processing circuitry 30 may also transmit information to external charging device that indicates any problems or errors with rechargeable power source 18 that may prevent rechargeable power source 18 from providing operational power to the components of IMD 14. In various examples, processing circuitry 30 may receive, through telemetry circuitry 36, instructions for algorithms, including formulas and/or values for constants to be used in the formulas, which may be used to determine the temperature of the housing 19 and/or exterior surface(s) of housing 19 of IMD 14 based on temperatures sensed by temperature sensor 39 located within IMD 14 during and after a recharging session performed on rechargeable power source 18.

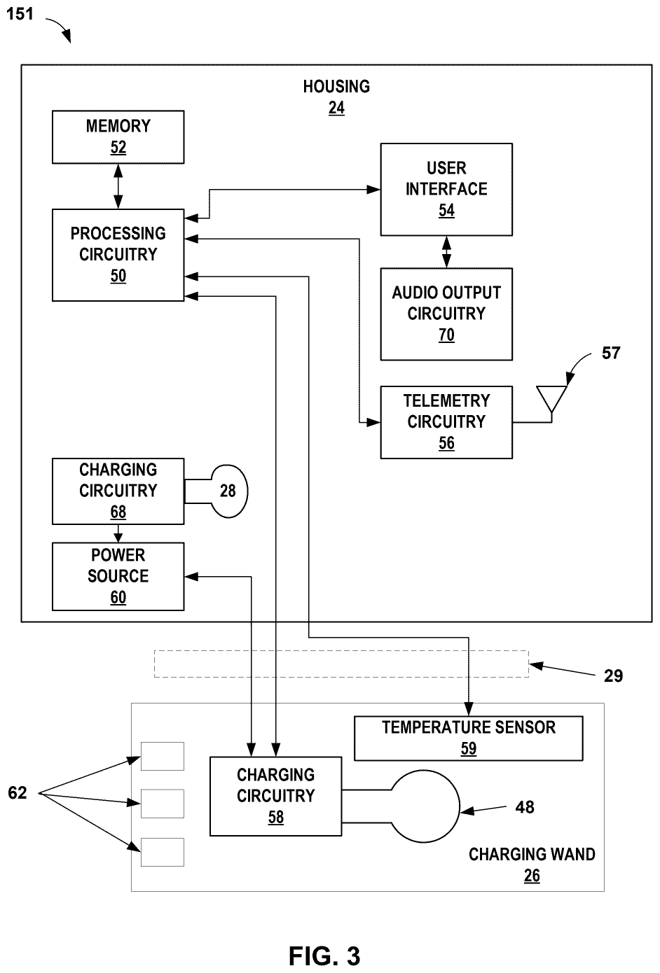

FIG. 3 is a block diagram of an example an external computing device 150 of FIGS. 1A and 1B. External charging device 151 in of FIG. 2 is an example of external computing device 150 described above in relation to FIGS. 1A and 1B. As described above, in some examples, external charging device 151 may be a hand-held device, in other examples, external charging device 151 may be a larger or a non-portable device. In addition, in other examples, external charging device 151 may be included as part of an external programmer or include functionality of an external programmer. As shown in the example of FIG. 3, external charging device 151 includes two separate components. Housing 24 encloses components such as a processing circuitry 50, memory 52, user interface 54, telemetry circuitry 56, power button, audio output circuitry 70 and power source 60. Charging head 26, also referred to as a charging wand 26, may include charging circuitry 58, temperature sensor 59, and coil 48. Housing 24 is electrically coupled to charging head 26 via charging cable 29. In some examples, housing 24 may also include charging circuitry 68 and coil 28, which is an example of coil 28 described above in relation to FIGS. 1A and 1B.

In some examples, separate charging wand 26 may facilitate positioning of coil 48 over coil 16 of IMD 14. Charging circuitry 68 and/or coil 28 may be integrated within housing 24 in other examples, as described above in relation to FIGS. 1A, 1B. In other examples, external charging device 151 may not include charging wand 26, but instead transfer energy to IMD 14 or other IMDs from primary coil 28. Memory 52 may store instructions that, when executed by processing circuitry 50, causes processing circuitry 50 and external charging device 151 to provide the functionality ascribed to external charging device 151 throughout this disclosure, and/or any equivalents thereof. Coil 48 and coil 28 may also be referred to as an antenna.

As described herein, external charging device 151 may be configured to determine a load on primary coil 28 and/or coil 48 in order to more quickly provide feedback to the user regarding alignment of primary and secondary coils without information from IMD 14. In some examples, charging circuitry 68 and/or charging circuitry 58 may include drive tank circuitry configured to provide current to a recharger tank circuit that may include a primary coil such as coil 28 or coil 48, respectively. In addition, charging circuitry 68 and/or charging circuitry 58 may include sense tank circuitry configured to sense one or more electrical parameters from the recharger tank circuit (e.g., Ptank, Itank), such as current and/or voltage in the recharger tank circuitry. Since changes to load on the coil, which may include recharger tank circuit, changes current and/or voltage representative of metal that may be within the magnetic field generated by the primary coil. Therefore, primary coil movement with respect to IMD 14 and/or other metal may cause the sense tank circuitry to output a change in one or more electrical parameter (e.g., the “load”) that can be interpreted as movement of the primary coil with respect to the secondary coil of IMD 14 and a change to inductive coupling efficiency.

External charging device 151 may also include one or more temperature sensors, illustrated as temperature sensor 59, similar to temperature sensor 39 of FIG. 2. As shown in FIG. 3, temperature sensor 59 may be disposed within charging head 26. For example, charging head 26 may include one or more temperature sensors positioned and configured to sense the temperature of coil 48 and/or a surface of the housing of charging head 26. In some examples, external charging device 151 may not include temperature sensor 59. In other examples, one or more temperature sensors of temperature sensor 59 may be disposed within housing 24, such as located to sense the temperature of primary coil 28 and/or charging circuitry 68.

In general, external charging device 151 comprises any suitable arrangement of hardware, alone or in combination with software and/or firmware, to perform the techniques ascribed to external charging device 151, and processing circuitry 50, user interface 54, telemetry circuitry 56, and charging circuitry 58 of external charging device 151, and/or any equivalents thereof. In various examples, external charging device 151 may include one or more processors, such as one or more microprocessors, DSPs, ASICs, FPGA s, or any other equivalent integrated or discrete logic circuitry, as well as any combinations of such components. Similar to IMD 14, components of external charging device 151 shown in FIG. 3 may be implemented as separate circuitry, or combined into one or more integrated circuits.

In other words, although processing circuitry 50, telemetry circuitry 56, charging circuitry 58, and temperature sensor 59 are described as separate modules, in some examples, processing circuitry 50, telemetry circuitry 56, charging circuitry 58, and/or temperature sensor 59 are functionally integrated. In some examples, processing circuitry 50, telemetry circuitry 56, charging circuitry 58, and/or temperature sensor 59 correspond to individual hardware units, such as ASICs, DSPs, FPGAs, or other hardware units. External charging device 151 also, in various examples, may include a memory 52, such as RAM, ROM, PROM, EPROM, EEPROM, flash memory, a hard disk, a CD-ROM, comprising executable instructions for causing the one or more processors to perform the actions attributed to them.

Memory 52 may store instructions that, when executed by processing circuitry 50, cause processing circuitry 50 and external charging device 151 to provide the functionality ascribed to external charging device 151 throughout this disclosure, and/or any equivalents thereof. For example, memory 52 may include instructions that cause processing circuitry 50 to control the power level used to charge IMD 14 in response to the determined temperatures for the housing/external surface(s) of IMD 14, as communicated from IMD 14, or instructions for any other functionality. Memory 52 may include a record of selected power levels, sensed temperatures, determined temperatures, or any other data related to charging rechargeable power source 18, described above in relation to FIG. 2. Memory 52 may also include instructions regarding the calculation of the load on a primary coil and how to provide feedback to the user regarding reduced alignment of primary and secondary coils and/or initiate communication with IMD 14.

Processing circuitry 50 may, when requested, transmit any stored data in memory 52 to another computing device for review or further processing, such as to servers 112 depicted in FIGS. 1A, 1B. Processing circuitry 50 may be configured to access memory, such as memory 32 of IMD 14 and/or memory 52 of external charging device 151, to retrieve information comprising instructions, formulas, and determined values for one or more constants.

User interface 54 may include buttons, a keypad, indicator lights, a microphone for voice commands, a display, such as a liquid crystal (LCD), light-emitting diode (LED), or cathode ray tube (CRT) and audio output circuitry 70. In some examples, user interface 54 may include a component to output vibration, such as a vibration motor, or may use audio output circuitry 70 to output vibration for haptic feedback. In some examples, user interface 54 may also connect to one or more skin electrodes 62, which may be configured to provide stimulation for haptic feedback when at least two electrodes are in contact with the skin of the patient. In any event, processing circuitry 50 may be configured to control user interface 54 to provide feedback to the user to improve alignment during recharge in response to changes to the detected load on the primary coil, such as coil 28 or coil 48.

In some examples, the display of user interface 54 may be a touch screen. As discussed in this disclosure, processing circuitry 50 may present and receive information relating to the charging of rechargeable power source 18 via user interface 54. For example, user interface 54 may indicate when charging is occurring, quality of the alignment between primary coil 28 or 48 and the secondary coil of the IMD, the selected power level, current charge level of rechargeable power source 18, duration of the current recharge session, duration of charging remaining, duration since charging started, anticipated remaining time of the charging session, sensed temperatures, or any other information. In some examples, user interface 54 may present a “speedometer” indicative of the calculated and/or estimated recharge coupling efficiency and charging speed. In some examples, processing circuitry 50 may receive some of the information displayed on user interface 54 from IMD 14, e.g., via communication circuitry such as telemetry circuitry 56. In some examples, user interface 54 may provide an indication to the user regarding the quality of alignment between coil 16, depicted in FIG. 2 and coil 48, based on one or more system metrics, such as the charge current to the battery of the IMD.

Processing circuitry 50 may also receive user input via user interface 54. The input may be, for example, in the form of pressing a button on a keypad or selecting an icon from a touch screen. The input may change programmed settings, start, or stop therapy, request starting or stopping a recharge session, a desired level of charging, or one or more statistics related to charging rechargeable power source 18 (e.g., the cumulative thermal dose). In this manner, user interface 54 may allow the user to view information related to the operation of IMD 14.

Charging circuitry 58 may include one or more circuits that generate an electrical signal, and an electrical current, within primary coil 48. Charging circuitry 58 may generate an alternating current of specified amplitude and frequency in some examples. In any case, charging circuitry 58 may be capable of generating electrical signals, and subsequent magnetic fields, to transmit various levels of power to IMD 14. In this manner, charging circuitry 58 may be configured to charge rechargeable power source 18 of IMD 14 with the selected power level.

Power source 60 may deliver operating power to the components of external charging device 151. Power source 60 may also deliver the operating power to drive primary coil 48 or primary coil 28 during the charging process. Power source 60 may include a battery and a power generation circuit to produce the operating power. In some examples, a battery of power source 60 may be rechargeable to allow extended portable operation. In other examples, power source 60 may draw power from a wired voltage source such as a consumer or commercial power outlet.

Telemetry circuitry 56 supports wireless communication between IMD 14 and external charging device 151 under the control of processing circuitry 50. Telemetry circuitry 56 may also be configured to communicate with another computing device via wireless communication techniques, or direct communication through a wired connection. In some examples, telemetry circuitry 56 may be substantially similar to telemetry circuitry 36 of IMD 14 described herein, providing wireless communication via an RF or proximal inductive medium. In some examples, telemetry circuitry 56 may include an antenna 57, which may take on a variety of forms, such as an internal or external antenna. Although telemetry circuitry 56 and 36 may each include dedicated antennas for communications between these devices, telemetry circuitry 56 and 36 may instead, or additionally, be configured to utilize inductive coupling from coils 16 and 48 to transfer data.

Examples of local wireless communication techniques that may be employed to facilitate communication between external charging device 151 and IMD 14 include radio frequency and/or inductive communication according to any of a variety of standard or proprietary telemetry protocols, or according to other telemetry protocols such as the IEEE 802.11x or Bluetooth specification sets. In this manner, other external devices may be capable of communicating with external charging device 151 without needing to establish a secure wireless connection.

In operation, processing circuitry 50, and or secondary processing circuitry 40, may control user interface 54 to provide information to a user about communication status, charging efficiency, therapy status of the IMD and so on. For example, processing circuitry 50 may determine whether communication circuitry, e.g., telemetry circuitry 56, has established a communication link with the power receiving device, device 10 and IMD 110 depicted in FIGS. 1A, 1B, and 2.

As described above in relation to FIGS. 1A, 1B, and 2, processing circuitry 50 may use any one or more system metrics to determine power transfer to device 10. In some examples, device 10 may send a signal indicating an amount of current output by the recharge circuitry of device 10. In other examples, processing circuitry 50 may calculate other system metrics, such as alignment of coil 28 to coil 16 of device 10 using any of several techniques, including heat calculations, temperature measurements, detection of metal, and so on. Processing circuitry may compare any of the calculated power transfer, power efficiency, alignment, IMD current, etc. to a threshold stored at memory 52.

Memory 52 may store several power coupling thresholds. A calculated power transfer metric (e.g., efficiency, magnitude of current, etc.) below a first threshold may indicate poor coupling. A power transfer metric above the first threshold but less than a second threshold may indicate “good” coupling. A power transfer metric above the second threshold but less than a third threshold may indicate “excellent” coupling, and so on. In some examples, responsive to determining that the power receiving device is receiving wireless power above the first threshold and receiving wireless power below the second threshold, processing circuitry 50 may control user interface 54 to display an indication of the quality of alignment, based on the magnitude of received wireless power. As described above in relation to FIGS. 1A-2, processing circuitry 50 may transmit information to the IMD to control delivered haptic stimulation based on the indication of the quality of alignment.

In some examples, processing circuitry 50 may also output an audio tone, pattern, and so on via audio output circuitry 70. In some examples, the audio pattern may be selectable by a user, such as the patient, based on the type of information that processing circuitry 50 is programmed to output. Some examples of patterns may include a warble tone, a distinctive musical sequence, and so on. For example, responsive to determining that the power receiving device is receiving wireless power above the first threshold and receiving wireless power below a second threshold, processing circuitry 50 may cause audio circuitry to output an audible alert with a first audible pattern. Processing circuitry 50 may cause audio output circuitry 70 to output other audio tones, patterns and so on for any of the conditions or states described herein, e.g., therapy on or off, and so on. In addition, as described herein, these thresholds may relate to the determined load associated with metal detected from the primary coil during energy transfer.

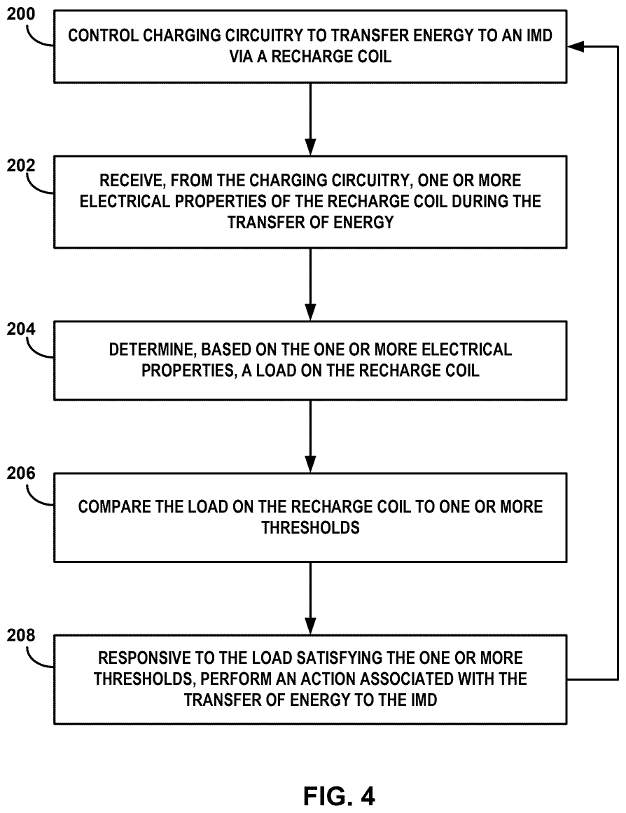

FIG. 4 is a flowchart illustrating an example operation of a recharging system for detecting metal load during energy transfer. The example of FIG. 4 is described with respect to external recharging device 151 of FIG. 3. However, the same techniques may be applied to system 100 of FIG. 1A or system 170 of FIG. 1B or any other described devices and systems. As shown in the example of FIG. 4, a power transmitting device, such as external charging device 151 may control charging circuitry to transfer energy an IMD 14 via a power transmitting antenna or recharge coil, e.g., primary coil 28 or primary coil 48 (200).

During the transfer of energy, processing circuitry 50 receives, from charging circuitry such as charging circuitry 58 or 68, one or more electrical properties of the recharge coil (202). The one or more electrical properties may be indicative of the metal load that is detected by the recharge coil. The charging circuitry may generate these electrical properties at a relatively fast frequency, such as once a second, four times a second, eight times a second, or even faster. This frequency may be one or more orders of magnitude faster than the communication frequency between external charging device 151 and IMD 14.

Processing circuitry 50 may then determine, based on the one or more electrical properties, a load on the recharge coil (204). This load may be a metric representative of the load or some other value that can be indicative of the metal loading on the primary coil. Processing circuitry 50 can then compare the load on the recharge coil to one or more thresholds (206). For example, one threshold may be a threshold that is representative of a low level of metal present such that IMD 14 cannot receive recharge power. In another example, the threshold may be a change threshold that relates to the amount of load change that has been detected that is representative of a significant alignment change between the recharge coil and the secondary coil of IMD 14. In another example, the threshold may be a target value which may be referred to as one or more bidirectional thresholds.

In response to the load satisfying one or more of these thresholds, processing circuitry 50 may perform an action associated with the transfer of energy to IMD 14 (208). Satisfaction of a threshold may be the load passing threshold, such as rising above, or dropping below, the threshold. This also may refer to exceeding a threshold. One action may be to immediately present feedback to the user to improve the alignment of the primary and secondary coils. Another action may be to initiate communication with IMD 14 in order to retrieve updated power transfer information that may confirm, or exclude, improper alignment. For example, metal loading may change while the coupling efficiency of the coupling remains sufficient without requesting the user to improve alignment. In any situation, the detected loading on the primary coil may be a trigger for external recharging device 151 to take action for additional information or presenting feedback to a user. The process of FIG. 4 may be applied to systems in which multiple recharge coils (e.g., multiple primary coils) are transferring energy to IMD 14, such as determining the load on each of the multiple coils, comparing the loads to the same or respective thresholds, and then performing an action based on one or more, or all, or the loads for the respective recharge coils satisfying the one or more thresholds. Such actions may include presenting feedback to the user to improve primary and secondary coil alignment or initiate communication with IMD to analyze power transfer information.

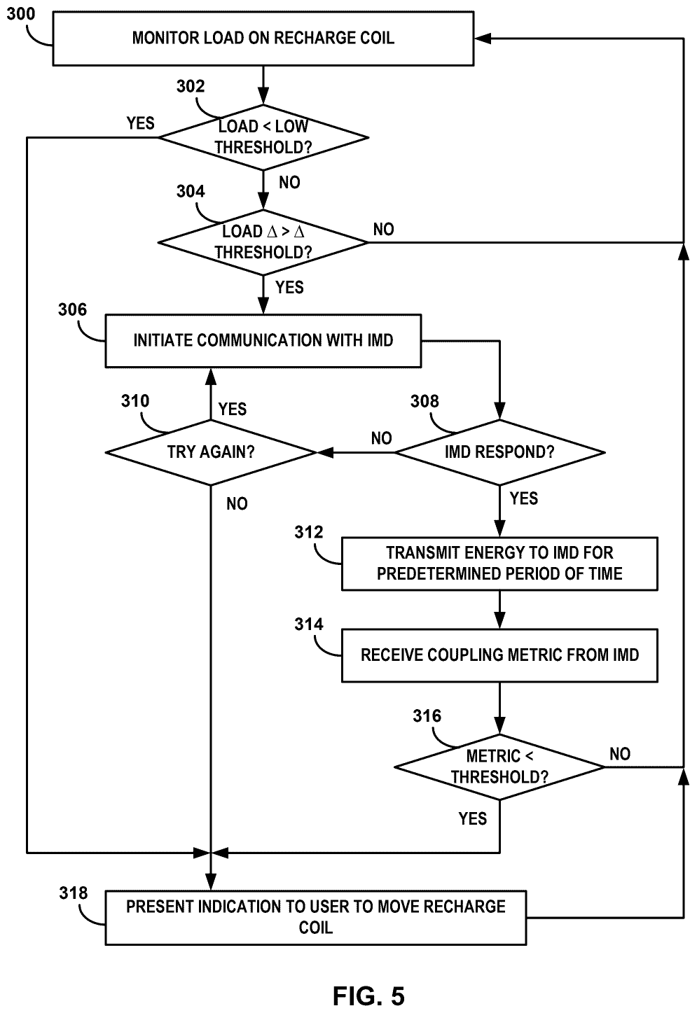

FIG. 5 is a flowchart illustrating an example operation of a recharging system for presenting an indication to a user based on metal load changes for one or more recharge coils. The example of FIG. 5 is described with respect to external recharging device 151 of FIG. 3. However, the same techniques may be applied to system 100 of FIG. 1A or system 170 of FIG. 1B or any other described devices and systems. As shown in the example of FIG. 5, a power transmitting device, such as external charging device 151 may control charging circuitry to transfer energy an IMD 14 via a power transmitting antenna or recharge coil, e.g., primary coil 28 or primary coil 48 and monitor the load on the recharge coil (300). This load monitoring may be similar to one or more aspects of the process of FIG. 4. The “load” is described as being determined herein, but the load may refer to one or more electrical property values or other metrics indicative of the load on the recharge coil. Although this load is described for a single coil such as primary coil 28 or primary coil 48, multiple coils may transmit power to IMD 14 at the same time. These multiple coils may be located in a single external charging device or be a part of a system in which different coils are positioned at different locations with respect to IMD 14.

Processing circuitry 50 may compare the load to a low threshold (302). If the load is below the low threshold (e.g., satisfies the low threshold) (“YES” branch of block 302), processing circuitry 50 may control user interface 54 to present an indication (e.g., feedback) to the user to move the recharge coil to improve alignment (318). If the load not below the low threshold (“NO” branch of block 302), processing circuitry 50 compares the load change to a change threshold (304). The load change may be the change in the load (e.g., percentage of change or magnitude of change over a period of time), and the change threshold may correspond to a predetermine percentage of magnitude of change that is representative of a significant change to the load that may be indicative of improper alignment. If the load change is less than the change threshold (“NO” branch of block 304), processing circuitry 50 continues to monitor the load on the recharge coil (300). When monitoring multiple recharge coils, processing circuitry 50 may determine the load of each recharge coil to the low threshold and the change threshold. The low threshold and change threshold may be the same for different recharge coils, or one or both of the low threshold and change thresholds may be different for different recharge coils. For monitoring multiple recharge coils, processing circuitry 50 may proceed to block 318 from block 302 in response to at least one of the multiple recharge coil load is below the low threshold or only in response to all recharge coil loads are below the low threshold, in some examples.

If the load change is greater than the change threshold (“YES” branch of block 304), processing circuitry 50 can attempt to initiate communication with IMD 14 (306). For monitoring multiple recharge coils, processing circuitry 50 may proceed to block 306 from block 304 in response to at least one of the multiple recharge coil has a load change greater than the change threshold or only in response to all recharge coils having a load change greater than the change threshold, in some examples.

The attempt to initiate communication may include external charging device 151 transmitting a signal to IMD 14 that requests the IMD transmit an updated recharge power transfer to the secondary coil of IMD 14, or some other indication of the coupling efficiency or alignment of the primary and secondary coils. If processing circuitry 50 does not receive a response from IMD 14 (“NO” branch of block 308), processing circuitry 50 may determine if another attempt at communication should be performed (310). For example, processing circuitry 50 may have instructions to attempt communication with IMD 14 a predetermined number of times, such as one, two, three, or more times. If processing circuitry 50 determines that another attempt should be made (“YES” branch of block 310″), processing circuitry 50 again attempts to initiate communication with IMD 14 (306). If processing circuitry 50 determines that another attempt should not be made (“NO” branch of block 310), processing circuitry 50 may control user interface 54 to present an indication (e.g., feedback) to the user to move the recharge coil to improve alignment (318). For multiple recharge coils, processing circuitry 50 may similarly control user interface 54 to present an indication to the user to move the recharge coils. This indication may include specific movements for respective recharge coils or a collective movement of the recharge coils. The movement instructions may be determined in order to achieve one or more results, such as achieving improved load for all coils or improved load for the coils as a whole, for example.

If processing circuitry 50 does receive a response from IMD 14 (“YES” branch of block 308), processing circuitry 50 controls charging circuitry to transmit energy to IMD 14 for a predetermined period of time using the recharge coil (312). This predetermined period of time may be selected in order for IMD 14 to calculate the coupling efficiency or power transfer from charging. Processing circuitry 50 can then receive a coupling metric from IMD 14 via telemetry (314). The coupling metric may be the power transfer value or other metric indicative of the coupling efficiency between the primary and secondary coils. In some examples, processing circuitry 50 may update the “speedometer” or other user interface presentation indicative of the charging speed or charging efficiency as feedback to the user. If the metric is greater than a threshold indicative of sufficient coupling efficiency (“NO” branch of block 316), processing circuitry 50 may continue to monitor the load on the recharge coil (300). If the metric is less than the threshold indicative of sufficient coupling efficiency (“YES” branch of block 316), processing circuitry 50 controls user interface 54 to present an indication (e.g., feedback) to the user to move the recharge coil to improve alignment (318). For multiple recharge coils, processing circuitry 50 may similarly determine the coupling efficiency or power transfer for all recharge coils or for each coil separately. The indication presented to the user may be an audible alert or haptic feedback that the user understands indicates that better alignment between the primary and secondary coils is requests. Alternatively, or additionally, processing circuitry 50 controls user interface 54 to display a graphic, text, or value that requests the user to re-align the coils. This re-alignment instruction may be for individual recharge coils for multiple coils or the recharge coils has a collective.

In some examples, processing circuitry 50 may update a “speedometer” or other indication of the charging speed or charging efficacy before returning to continue to monitor the load in step 300. For example, using updated load data and/or other information from IMD 14, processing circuitry 50 may recalculate the indication of charging speed or efficiency. This may occur at any point within the technique of FIG. 5 as processing circuitry 50 obtains updated load information.

In some examples, the technique of FIG. 5 may also, or alternatively, detect high levels of metal load which may be indicative of other metal in or near the patient, such as an unexpected portion of IMD 14 (e.g., header of the IMD or asymmetrical portions) or another structure such as an orthopedic implant. For example, prior to decision block 302, processing circuitry 50 may determine if the load is greater than a high threshold. If the lead is greater than the high threshold (e.g., satisfies the high threshold), processing circuitry 50 may control user interface 54 to present an indication (e.g., feedback) to the user to move the recharge coil to improve alignment (318). If the load not above the high threshold, processing circuitry 50 proceeds to decision block 302. The high threshold value may be based on experimental values or a predetermined percentage or amount over typical metal loads detecting during known proper alignment between the primary and secondary coils. In this manner, processing circuitry 50 may be configured to determine, based on comparison of the load to the one or more thresholds, that the load is above below the one or more thresholds which indicates that the recharge coil is detecting excess metal which indicates improper alignment to a secondary coil of the IMD. This process of detecting high levels of metal load may similarly be performed for multiple recharge coils that are energized for recharging the IMD.

The following examples are described herein.

Example 1. An external charging device, the device comprising: a recharge coil configured to transfer energy to an implantable medical device (IMD) and detect metal loading; charging circuitry coupled to the recharge coil and configured to determine one or more electrical properties of the recharge coil during the transfer of energy; and processing circuitry configured to: determine, based on the one or more electrical properties, a load on the recharge coil; compare the load on the recharge coil to one or more thresholds; and responsive to the load satisfying the threshold, perform an action associated with the transfer of energy to the IMD.

Example 2. The device of example 1, wherein the processing circuitry is configured to determine, based on the comparison of the load to the one or more thresholds, that the load is dropped below the one or more thresholds which indicates that the recharge coil is insufficiently aligned to a secondary coil of the IMD, and wherein the action comprises, controlling a user interface to display a representation indicative of improper coupling between the recharge coil and the secondary coil.

Example 3. The device of any of examples 1 or 2, wherein the processing circuitry is configured to determine, based on the comparison of the load to the one or more thresholds, that the load is above below the one or more thresholds which indicates that the recharge coil is detecting excess metal which indicates improper alignment to a secondary coil of the IMD, and wherein the action comprises, controlling a user interface to display a representation indicative of improper coupling between the recharge coil and the secondary coil.

Example 4. The device of any of examples 1 through 3, wherein the action comprises initiating communication with the IMD.

Example 5. The device of example 4, wherein the processing circuitry is configured to, responsive to initiating communication with the IMD, receive coupling information from the IMD indicative of coupling of the recharge coil to a secondary coil of the IMD.

Example 6. The device of any of examples 4 and 5, wherein the processing circuitry is configured to, responsive to communication initiation with the IMD, control the charging circuitry to charge for a period of time, wherein the coupling information is representative of coupling efficiency for the period of time.

Example 7. The device of any of examples 1 through 6, wherein the processing circuitry is configured to, responsive to determining that no communication has been initiated between the device and the IMD, control a user interface to present an indication to a user that coupling with the IMD is below a threshold.

Example 8. The device of any of examples 1 through 7, wherein the processing circuitry is configured to control a user interface to present an indication to a user that coupling with the IMD is below a threshold.

Example 9. The device of any of examples 1 through 8, wherein the processing circuitry is configured to estimate a recharge coupling efficiency based on the load on the recharge coil.

Example 10. The device of example 9, wherein the processing circuitry is configured to control a user interface to display a representation indicative of the estimated recharge coupling efficiency.

Example 11. The device of any of examples 1 through 10, wherein the IMD is configured to deliver tibial nerve stimulation.

Example 12. A method comprising: transferring, by a recharge coil, energy to an implantable medical device (IMD), wherein the recharge coil is configured to detect metal loading; determining, by charging circuitry coupled to the recharge coil, one or more electrical properties of the recharge coil during the transfer of energy; determining, by processing circuitry and based on the one or more electrical properties, a load on the recharge coil; comparing, by the processing circuitry, the load on the recharge coil to one or more thresholds; and responsive to the load satisfying the threshold, performing, by the processing circuitry, an action associated with the transfer of energy to the IMD.

Example 13. The method of example 12, further comprising determining, based on the comparison of the load to the one or more thresholds, that the load is dropped below the one or more thresholds which indicates that the recharge coil is insufficiently aligned to a secondary coil of the IMD, and wherein performing the action comprises controlling a user interface to display a representation indicative of improper coupling between the recharge coil and the secondary coil.

Example 14. The method of any of examples 12 or 13, further comprising determining, based on the comparison of the load to the one or more thresholds, that the load is above below the one or more thresholds which indicates that the recharge coil is detecting excess metal which indicates improper alignment to a secondary coil of the IMD, and wherein the action comprises, controlling a user interface to display a representation indicative of improper coupling between the recharge coil and the secondary coil.

Example 15. The method of any of examples 12 through 14, wherein performing the action comprises initiating communication with the IMD.

Example 16. The method of example 15, further comprising, responsive to initiating communication with the IMD, receiving coupling information from the IMD indicative of coupling of the recharge coil to a secondary coil of the IMD.

Example 17. The method of any of examples 15 and 16, further comprising, responsive to communication initiation with the IMD, controlling the charging circuitry to charge for a period of time, wherein the coupling information is representative of coupling efficiency for the period of time.

Example 18. The method of any of examples 12 through 17, further comprising, responsive to determining that no communication has been initiated between the device and the IMD, controlling a user interface to present an indication to a user that coupling with the IMD is below a threshold.

Example 19. The method of any of examples 12 through 18, further comprising controlling a user interface to present an indication to a user that coupling with the IMD is below a threshold.

Example 20. The method of any of examples 12 through 19, further comprising estimating a recharge coupling efficiency based on the load on the recharge coil.

Example 21. The method of example 20, further comprising controlling a user interface to display a representation indicative of the estimated recharge coupling efficiency.

Example 22. A non-transitory computer-readable medium comprising instructions that, when executed, controls the processing circuitry to: control a recharge coil to transfer energy to an implantable medical device (IMD), wherein the recharge coil is configured to detect metal loading, wherein charging circuitry coupled to the recharge coil determines one or more electrical properties of the recharge coil during the transfer of energy; determine, based on the one or more electrical properties, a load on the recharge coil; compare the load on the recharge coil to one or more thresholds; and responsive to the load satisfying the threshold, perform an action associated with the transfer of energy to the IMD.

In one or more examples, the functions described above may be implemented in hardware, software, firmware, or any combination thereof. For example, the various components of FIGS. 2 and 3, such as processing circuitry 30, therapy and sensing circuitry 34, telemetry circuitry 36, processing circuitry 50 and so on may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, A SICs, FPGAs, or other equivalent integrated or discrete logic circuitry. Accordingly, the term “processor,” and processing circuitry as used herein may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

What is claimed is:

1. An external charging device, the device comprising:
a recharge coil configured to transfer energy to an implantable medical device (IMD) and detect metal loading;
charging circuitry coupled to the recharge coil and configured to determine one or more electrical properties of the recharge coil during the transfer of energy; and
processing circuitry configured to:
determine, based on the one or more electrical properties, a load on the recharge coil;
compare the load on the recharge coil to one or more thresholds; and
responsive to the load satisfying the threshold, perform an action associated with the transfer of energy to the IMD.

2. The device of claim 1, wherein the processing circuitry is configured to determine, based on the comparison of the load to the one or more thresholds, that the load is dropped below the one or more thresholds which indicates that the recharge coil is insufficiently aligned to a secondary coil of the IMD, and wherein the action comprises, controlling a user interface to display a representation indicative of improper coupling between the recharge coil and the secondary coil.

3. The device of claim 1, wherein the processing circuitry is configured to determine, based on the comparison of the load to the one or more thresholds, that the load is above below the one or more thresholds which indicates that the recharge coil is detecting excess metal which indicates improper alignment to a secondary coil of the IMD, and wherein the action comprises, controlling a user interface to display a representation indicative of improper coupling between the recharge coil and the secondary coil.

4. The device of claim 1, wherein the action comprises initiating communication with the IMD.

5. The device of claim 4, wherein the processing circuitry is configured to, responsive to initiating communication with the IMD, receive coupling information from the IMD indicative of coupling of the recharge coil to a secondary coil of the IMD.

6. The device of claim 4, wherein the processing circuitry is configured to, responsive to communication initiation with the IMD, control the charging circuitry to charge for a period of time, wherein the coupling information is representative of coupling efficiency for the period of time.

7. The device of claim 1, wherein the processing circuitry is configured to, responsive to determining that no communication has been initiated between the device and the IMD, control a user interface to present an indication to a user that coupling with the IMD is below a threshold.

8. The device of claim 1, wherein the processing circuitry is configured to control a user interface to present an indication to a user that coupling with the IMD is below a threshold.

9. The device of claim 1, wherein the processing circuitry is configured to estimate a recharge coupling efficiency based on the load on the recharge coil.

10. The device of claim 9, wherein the processing circuitry is configured to control a user interface to display a representation indicative of the estimated recharge coupling efficiency.

11. The device of claim 1, wherein the IMD is configured to deliver tibial nerve stimulation.

12. A method comprising:
transferring, by a recharge coil, energy to an implantable medical device (IMD), wherein the recharge coil is configured to detect metal loading;
determining, by charging circuitry coupled to the recharge coil, one or more electrical properties of the recharge coil during the transfer of energy;
determining, by processing circuitry and based on the one or more electrical properties, a load on the recharge coil;
comparing, by the processing circuitry, the load on the recharge coil to one or more thresholds; and
responsive to the load satisfying the threshold, performing, by the processing circuitry, an action associated with the transfer of energy to the IMD.

13. The method of claim 12, further comprising determining, based on the comparison of the load to the one or more thresholds, that the load is dropped below the one or more thresholds which indicates that the recharge coil is insufficiently aligned to a secondary coil of the IMD, and wherein performing the action comprises controlling a user interface to display a representation indicative of improper coupling between the recharge coil and the secondary coil.

14. The method of claim 12, further comprising determining, based on the comparison of the load to the one or more thresholds, that the load is above below the one or more thresholds which indicates that the recharge coil is detecting excess metal which indicates improper alignment to a secondary coil of the IMD, and wherein the action comprises, controlling a user interface to display a representation indicative of improper coupling between the recharge coil and the secondary coil.

15. The method of claim 12, wherein performing the action comprises initiating communication with the IMD.

16. The method of claim 15, further comprising, responsive to initiating communication with the IMD, receiving coupling information from the IMD indicative of coupling of the recharge coil to a secondary coil of the IMD.

17. The method of claim 15, further comprising, responsive to communication initiation with the IMD, controlling the charging circuitry to charge for a period of time, wherein the coupling information is representative of coupling efficiency for the period of time.

18. The method of claim 12, further comprising, responsive to determining that no communication has been initiated between the device and the IMD, controlling a user interface to present an indication to a user that coupling with the IMD is below a threshold.

19. The method of claim 12, further comprising controlling a user interface to at least one of:
present an indication to a user that coupling with the IMD is below a threshold, or display a representation indicative of the estimated recharge coupling efficiency.

20. A non-transitory computer-readable medium comprising instructions that, when executed, controls the processing circuitry to:
control a recharge coil to transfer energy to an implantable medical device (IMD), wherein the recharge coil is configured to detect metal loading, wherein charging circuitry coupled to the recharge coil determines one or more electrical properties of the recharge coil during the transfer of energy;
determine, based on the one or more electrical properties, a load on the recharge coil;
compare the load on the recharge coil to one or more thresholds; and
responsive to the load satisfying the threshold, perform an action associated with the transfer of energy to the IMD.
